(19) United States
(12) Patent Application Publication
(10) Pub. No.: US 2018/0230458 A1
(43) Pub. Date: Aug. 16, 2018

(54) METHOD AND COMPOSITIONS FOR DETECTING PATHOGENIC ORGANISMS

(71) Applicant: EPICENTRE TECHNOLOGIES CORPORATION, Madison, WI (US)

(72) Inventors: Haiying Li Grunenwald, Madison, WI (US); Stephen Paul Bruinsma, Madison, WI (US); Anupama Khanna, Madison, WI (US); Ramesh Vaidyanathan, Madison, WI (US)

(21) Appl. No.: 15/524,780

(22) PCT Filed: Nov. 12, 2015
(86) PCT No.: PCT/US2015/060296

§ 371 Date: May 5, 2017

Related U.S. Application Data
(60) Provisional application No. 62/081,193, filed on Nov. 18, 2014.

(51) Int. Cl. C12N 15/10; C12Q 1/6806; C12Q 1/6869; C12Q 1/6876; C40B 20/04; C40B 70/00; C40B 50/10; C40B 50/04; C40B 40/12

(52) U.S. Cl. CPC C12N 15/1058; C12Q 1/6806; C12Q 1/6869; C12Q 1/6876; C40B 20/04; C40B 70/00; C12Q 2563/149; C40B 50/04; C40B 40/12; C12Q 2535/122; C12Q 2537/159; C12Q 2563/131; C40B 50/10

(57) ABSTRACT

Some embodiments of the present invention relate to the enrichment of non-host nucleic acids in a mixture of host and non-host nucleic acids. Some embodiments include methods for detecting pathogenic organisms from a nucleic acid sample comprising host nucleic acids and nucleic acids indicative of the pathogenic organism.

### RELATED APPLICATIONS

This application claims the benefit of U.S. Provisional Application No. 62/081,193 filed Nov. 18, 2014 entitled “METHOD AND COMPOSITIONS FOR DETECTING PATHOGENIC ORGANISMS” which is incorporated by reference herein in its entirety.

### FIELD OF THE INVENTION

Some embodiments of the present disclosure relate to the enrichment of non-host nucleic acids in a mixture of host and non-host nucleic acids. Some embodiments include methods for detecting pathogenic organisms from a nucleic acid sample comprising host nucleic acids and nucleic acids indicative of the pathogenic organism.

### BACKGROUND OF THE DISCLOSURE

The falling cost of DNA sequencing means that sample quality, rather than expense, is now a blocking issue for many infectious disease genome sequencing projects. Pathogen genomes are generally very small relative to that of their human host, and are typically haploid in nature. Therefore, even a modest number of nucleated human cells present in infectious disease samples may result in the pathogen DNA representation being dwarfed relative to the host human DNA. This difference in representation poses a significant challenge to achieving adequate sequence coverage of the pathogen genome in a cost-effective manner. Separation of host and pathogen cells prior to DNA extraction can be difficult or inconvenient, particularly in field settings common to clinical trials in developing countries. The increasing use of genome-wide association studies to determine the genetic basis of important infectious disease phenotypes, such as drug resistance, requires sequencing or genotyping hundreds to thousands of pathogen isolates, making a shortage of quality specimens an acute problem.

Existing methods for dealing with human DNA contamination in infectious disease samples typically require significant time, money, or special handling of samples at the time of collection. Thus, there exists a need for improved methods for sequencing pathogen DNA in samples that contain host or other contaminating DNA.

### SUMMARY OF THE DISCLOSURE

Some embodiments of the methods, compositions and uses provided herein include a method for the enrichment of non-host RNAs in a nucleic acid sample comprising host RNAs and non-host RNAs, comprising: (a) obtaining a plurality of capture probes, wherein each capture probe comprises an affinity tag and a nucleic acid complementary to a host RNA; (b) contacting the nucleic acid sample with the plurality of capture probes; and (c) removing capture probes hybridized to the host RNAs, thereby obtaining a population of nucleic acids enriched for non-host RNAs.

In some embodiments, the plurality of capture probes comprises capture probes prepared by a method comprising: (i) obtaining single-stranded target nucleic acids; (ii) obtaining double-stranded target nucleic acids from the single-stranded target nucleic acids, wherein the double-stranded target nucleic acids comprise an RNA polymerase promoter; and (iii) contacting the double-stranded nucleic acids with an RNA polymerase to obtain RNAs complementary to the single-stranded target nucleic acids.

In some embodiments, the double-stranded target nucleic acids comprise cDNA.

In some embodiments, step (ii) comprises linking the double-stranded target nucleic acids with a primer comprising the RNA polymerase promoter or complement thereof.

In some embodiments, the double-stranded target nucleic acids comprise RNA.

In some embodiments, step (ii) comprises linking the single-stranded target nucleic acids with a primer comprising the RNA polymerase promoter or a complement thereof.

In some embodiments, step (ii) comprises linking the single-stranded nucleic acids with an adapter primer, and hybridizing a primer comprising the RNA polymerase promoter or a complement thereof to the adapter primer.

In some embodiments, step (ii) comprises contacting the single-stranded target nucleic acids with a reverse transcriptase.

In some embodiments, the reverse transcriptase is selected from the group consisting of Moloney murine leukemia virus (MMLV) reverse transcriptase, and avian myeloblastosis virus (AMV) reverse transcriptase.

In some embodiments, the plurality of capture probes comprises capture probes prepared by a method comprising: (i) linking the double-stranded nucleic acids fragments with a primer comprising an RNA polymerase promoter; (ii) amplifying the double-stranded nucleic acids fragments with an RNA polymerase; and (iii) fragmenting the RNA probes.

In some embodiments, the double-stranded nucleic acids comprise genomic DNA.

In some embodiments, the plurality of capture probes comprises capture probes prepared by a method comprising: (i) inserting a plurality of transposons into target nucleic acids, wherein insertion of the transposon into target nucleic acid by the transposome complex fragments the target nucleic acid and simultaneously inserts an RNA polymerase promoter; and (ii) amplifying the double-stranded nucleic acids fragments with an RNA polymerase.

In some embodiments, the transposon is selected from the group consisting of Mu, Mu E392Q, Tn5, RAG, and Tn552.

In some embodiments, wherein the transposons comprise a fragmentation site, and the method further comprises fragmenting the target nucleic acids at the fragmentations sites.

In some embodiments, the RNA polymerase is selected from the group consisting of T7 RNA polymerase, T3 RNA polymerase, and SP6 RNA polymerase.

In some embodiments, the plurality of capture probes comprises capture probes prepared by amplification to obtain the capture probes comprising affinity tags.

In some embodiments, the affinity tag is selected from the group consisting of an antibody, an antibody fragment, a receptor protein, a hormone, biotin, streptavidin, a His tag, and digoxin.

In some embodiments, the nucleic acid sample further comprises DNA. Some embodiments also include depleting DNA from the nucleic acid sample.

Some embodiments also include depleting polyadenylated RNAs from the nucleic acid sample.

Some embodiments also include contacting the nucleic acid sample with a plurality of capture probes comprising poly-T nucleic acids. In some embodiments, the plurality of capture probes comprising poly-T nucleic acids are attached to a substrate. In some embodiments, the substrate comprises beads.

In some embodiments, the capture probes are prepared from a source selected from the group consisting of a cell, a cell-line, a tissue, and an organ.

In some embodiments, the plurality of capture probes comprises capture probes complementary to RNAs selected from the group consisting of messenger RNAs, ribosomal RNAs, mitochondrial RNAs, transfer RNAs, micro RNAs, and small inhibitory RNAs. In some embodiments, the host RNAs comprise eukaryotic RNAs. In some embodiments, the host RNAs comprise mammalian RNAs. In some embodiments, the host RNAs comprise human RNAs. In some embodiments, the host RNAs comprise plant RNAs. In some embodiments, the host RNAs comprise prokaryotic RNAs. In some embodiments, the host RNAs comprise bacterial RNAs. In some embodiments, the non-host RNAs are derived from a pathogenic organism or virus. In some embodiments, the non-host RNAs are selected from the group consisting of eukaryotic RNAs, prokaryotic RNAs, viral RNAs, degraded RNAs, ancient RNAs, and artificial RNAs.

In some embodiments, the plurality of capture probes is linked to a substrate. In some embodiments, the substrate comprises beads. In some embodiments, the substrate comprises a planar surface.

In some embodiments, the plurality of capture probes is in solution.

In some embodiments, the non-host RNAs are enriched in the population of nucleic acids enriched for non-host RNAs compared to the nucleic acid sample by at least about 10-fold, 50-fold, 80-fold, 100-fold, and 200-fold.

Some embodiments of the methods, compositions and uses provided herein include a nucleic acid sequencing library comprising nucleic acids obtained by any one of the foregoing methods.

Some embodiments of the methods, compositions and uses provided herein include a method for detecting the presence of a pathogen in a sample comprising: obtaining a nucleic acid sample comprising host RNAs and non-host RNAs from the sample, wherein the pathogen comprises the non-host RNAs; enriching the nucleic acid sample for the non-host RNAs according to any one of the foregoing methods; and detecting the presence of the non-host RNAs in the enriched nucleic acid sample. In some embodiments, detecting the presence of the non-host RNAs comprises obtaining sequence information from the enriched nucleic acid sample.

### BRIEF DESCRIPTION OF THE DRAWINGS

FIG. 1 shows an exemplary graph of percentage alignment to E. coli. transcriptome of sequences obtained from samples comprising E. coli RNAs and human RNAs in which samples were treated using a Ribo-Zero™ kit (Epicentre, Madison, Wis.), or a Ribo-Zero™ kit with prepared biotinylated capture probes (aRNA), or fragmented. Numbers above each column depict fold-enrichment.

FIG. 2 shows exemplary graphs of percentage alignment and fold enrichment for non-host RNAs in various samples that included 0.2% non-host RNAs or 1% non-host RNAs. The graphs depict a calculation of the level of enrichment needed to give the indicated percent of observed pathogen reads for samples starting with 0.2% or 1.0% non-host RNA.

FIG. 3 shows an exemplary graph of percentage alignment and fold enrichment of non-host RNA (E. coli) in a host sample using various depletion methods, including the method disclosed herein.

FIG. 4 shows a graph of percentage alignment and fold enrichment of non-host RNA (E. coli) in a host sample using various depletion methods, including the method disclosed herein.

### DETAILED DESCRIPTION

Some embodiments of the present disclosure relate to the enrichment of non-host nucleic acids in a mixture of host and non-host nucleic acids. Some embodiments include methods for detecting pathogenic organisms from a nucleic acid sample comprising host nucleic acids and nucleic acids indicative of a pathogenic organism. In some embodiments, host nucleic acids are depleted from a nucleic acid sample comprising host and non-host nucleic acids.

In some embodiments, the non-host nucleic acids comprise a minor fraction of the total nucleic acids in a nucleic acid sample. In some embodiments, host nucleic acids are depleted from a nucleic acid sample, thereby enriching the nucleic acid sample with non-host nucleic acids. In some embodiments, host nucleic acids are depleted from a nucleic acid sample by contacting the nucleic acid sample with capture probes to remove host nucleic acids. In some embodiments, the capture probes include nucleic acids comprising sequences complementary to host RNAs. In some embodiments, capture probes comprise antisense RNAs (aRNAs). In some embodiments, the capture probes comprise affinity tags to facilitate removal of capture probes hybridized to host RNAs.

Some embodiments include the preparation of capture probes from target nucleic acids, such as host RNAs. Target nucleic acids can include a complex mixture of RNAs characteristic of a host, such as the transcriptome of a host. Advantageously, use of capture probes that include nucleic acids comprising sequences complementary to host RNAs and generated from a complex mixture of host RNAs can enrich for nucleic acids with unknown sequences. In other words, a nucleic acid sample can be enriched for certain nucleic acids with unknown sequences. In some embodiments, host RNA depletion preferentially removes highly expressed host RNAs, therefore remaining host RNA is normalized in which non-coding RNAs and low expressors are preferentially enriched.

In some embodiments, host nucleic acids, such as host RNAs are further depleted from a nucleic acid sample using capture probes generated against certain species of host RNAs. In some embodiments, capture probes comprising sequences complementary to ribosomal RNAs, tRNAs, polyadenylated RNAs, mitochondrial RNAs and other non-polyadenylated RNAs can be prepared and/or utilized to deplete host RNAs from a nucleic acid sample.

In some embodiments, host DNA is depleted from a nucleic acid sample. In some embodiments, capture probes comprising genomic DNA sequences can be prepared and/or utilized to deplete host DNA from a nucleic acid sample. In some embodiments, capture probes can be prepared by in vitro transcription of fragmented genomic DNA. In some embodiments, genomic DNA can be fragmented by insertion of transposons. In some embodiments, the transposons comprise fragmentation sites. In some embodiments, inserted transposons include primer sites to generate capture probes.

As used herein, “host” includes any organism that harbors another organism, such as a pathogen, parasite, commensal organism, or symbiont. Hosts may be human or non-human animals or (e.g., mammals or plants). In some embodiments, a host is eukaryotic. In some embodiments, a host is mammalian. In some embodiments, a host is human. In some embodiments, a host is a plant. In some embodiments, a host is prokaryotic. In some embodiments, a non-host includes a pathogenic organism or virus. In some embodiments, a non-host is eukaryotic. In some embodiments, a non-host is prokaryotic. In some embodiments, a non-host is viral. In some embodiments, a non-host comprises degraded nucleic acids. In some embodiments, a non-host comprises ancient nucleic acids. In some embodiments, a non-host comprises artificial nucleic acids. As used herein, artificial nucleic acids, such as artificial RNAs, can include nucleic acids having non-naturally occurring sequences, and can include nucleic acids comprising synthetic nucleotides. As used herein, ancient nucleic acids, such as ancient RNAs, can include nucleic acids obtained from a source that has not lived for at least about 6 months, 12 months, 5 years, 10 years, 100 years, 500 years or any range between the foregoing. In some embodiments, ancient nucleic acids include nucleic acids obtained from a host that has not lived for at least about 6 months, 12 months, 5 years, 10 years, 100 years, 500 years, 1000 years, 5000 years, 10,000 years 50,000 years or any range between the foregoing.

As used herein, “target nucleic acid” includes host nucleic acids. In some embodiments, target nucleic acids can be used to generate capture probes, such as antisense RNAs (aRNAs). In some embodiments, a target nucleic acid includes RNA of a host.

### Enriching for Non-Host Nucleic Acids

Some embodiments of the methods and compositions provided herein include methods for the enrichment of non-host nucleic acids, such as non-host RNAs, in a nucleic acid sample comprising host nucleic acids, such as host RNAs, and non-host nucleic acids, such as non-host RNAs. In some embodiments, the host nucleic acids, such as host RNAs include eukaryotic RNAs, mammalian RNAs, human RNAs, plant RNAs, prokaryotic RNAs, or bacterial RNAs. In some embodiments, non-host nucleic acids, such as non-host RNAs, are derived from a pathogenic organism or virus. In some embodiments, the non-host RNAs include eukaryotic RNAs, prokaryotic RNAs, viral RNAs, degraded RNAs, ancient RNAs, or artificial RNAs.

Some embodiments include obtaining a plurality of capture probes. In some embodiments, the capture probes comprise nucleic acids which include sequences complementary to host nucleic acids, such as host RNAs. In some embodiments, the capture probes comprise RNA. In some embodiments, the capture probes comprise antisense RNA (aRNA). In some embodiments, the capture probes comprise affinity tags. Embodiments of methods for preparing capture probes are provided herein.

Some embodiments include contacting the nucleic acid sample with the plurality of capture probes. In some embodiments, the capture probes hybridize to host RNAs in the nucleic acid sample. The hybridization complexes can be removed from the nucleic acid sample with an appropriate capture system, thereby obtaining a population of nucleic acids enriched for non-host RNAs. As used herein, “hybridization”, “hybridizes” or “capable of hybridizing” includes forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

In some embodiments, a nucleic acid sample can be enriched for non-host nucleic acids, such as non-host RNAs, relative to a non-enriched initial nucleic acids sample by about or at least about 2, 5, 10, 20, 30, 40, 50, 100, 200, 300, 400, 500, 600, 700, 800, 900, or 1000 fold or any range derivable therein. In some embodiments, non-host nucleic acids, such as non-host RNAs, are enriched in a population of nucleic acids compared to an initial non-enriched nucleic acid sample by a factor of at least 10-fold, 50-fold, 80-fold, 100-fold, or 200-fold. In some embodiments, host nucleic acids, such as host RNAs, are depleted from a nucleic acid sample relative to a non-depleted initial sample by about or at least about 5%, 10%, 20%, 30%, 40%, 50%, 60%, 70%, 80%, 90%, 95%, 99%, 100%, or any range derivable therein.

In some embodiments, the nucleic acid sample further comprises DNA. Some embodiments include depleting DNA from the nucleic acid sample. Methods of depleting DNA or separating DNA from RNA are well known to those skilled in the art. In some embodiments, the sample is incubated with DNase. In some embodiments, DNA is extracted using an acid phenol:chloroform extraction in which acid phenol:chloroform (e.g., 5:1 phenol:CHCl₃; pH 4.7) extraction partitions DNA to the organic phase and the RNA remains in the aqueous phase and can be subsequently recovered by precipitation. In some embodiments, DNA is separated from RNA using lithium chloride precipitation. In some embodiments, the nucleic acid sample is further contacted with DNase to remove remaining DNA.

Some embodiments include depleting polyadenylated RNAs from the nucleic acid sample. Methods for isolating polyadenylated mRNA from a sample are well known in the art. For example, a common method for isolating polyadenylated mRNA comprises hybridizing the polyadenylated mRNA to a poly(T) oligonucleotide. Typically, the poly(T) oligonucleotide is attached to a surface, such as a column or a bead. After the polyadenylated mRNA is hybridized to the poly(T) oligonucleotide, it can be separated from the sample. For example, if the polyadenylated mRNA is hybridized to the poly(T) oligonucleotide immobilized on a magnetic bead. The beads may then be separated from the sample using a magnet.

Some embodiments include depleting ribosomal RNAs from the nucleic acid sample. In some embodiments, it may be desirable to deplete eukaryotic rRNA, bacterial rRNA, or both. In some embodiments, eukaryotic rRNA may hybridize with one or more oligonucleotides complementary to at least a portion of one or more of the 5S rRNA, 5.8S rRNA, 17S rRNA, 18S rRNA, or 28S rRNA. In some embodiments, bacterial rRNA may be hybridized with one or more oligonucleotides complementary to at least a portion of one or more of the 5S rRNA, 16S rRNA or 23S rRNA. The hybridization complexes are then removed from the sample with an appropriate capture system. In some embodiments, the oligonucleotides are in solution. In some embodiments, the oligonucleotides are immobilized on a surface, which enables the removal of the hybridization complexes.

In some embodiments, the capture probes are in solution. In some embodiments, capture probes are immobilized on a substrate. In some embodiments, the capture probe is immobilized on a substrate through an affinity tag and a binding partner associated with the substrate. In some embodiments, the substrate comprises beads. In some embodiments, the substrate comprises a planar surface.

Some embodiments include nucleic acid sequencing libraries comprising nucleic acids obtained by any of the methods for enriching non-host RNAs provided herein.

Some embodiments include methods for detecting the presence of a pathogen in a sample comprising: obtaining a nucleic acid sample comprising host RNAs and non-host RNAs from the sample, wherein the pathogen comprises the non-host RNAs; enriching the nucleic acid sample for the non-host RNAs according to any of the methods for enriching non-host RNAs provided herein and detecting the presence of the non-host RNAs in the enriched nucleic acid sample. In some embodiments, detecting the presence of the non-host RNAs comprises obtaining sequence information from the enriched nucleic acid sample.

In some embodiments, non-host nucleic acids are indicative of certain bacteria (e.g., Gram-negative bacteria or Gram-positive bacteria), mycobacteria, mycoplasma, fungi, and parasitic cells. In some embodiments, non-host nucleic acids are indicative of a pathogen, a parasite, a commensal organism, or a symbiont.

In some embodiments, non-host nucleic acids are indicative of Plasmodium vivax, Chlamydia trachomatis, Trypanosoma cruzi, and Wolbachia. In some embodiments, non-host nucleic acids are indicative of Plasmodium falciparum, Plasmodium ovale, and Plasmodium malariae.

In some embodiments, non-host nucleic acids are indicative of certain gram-negative bacteria. In some embodiments, gram-negative bacteria include bacteria of the genera, Salmonella, Escherichia, Chlamydia, Klebsiella, Haemophilus, Pseudomonas, Proteus, Neisseria, Vibro, Helicobacter, Brucella, Bordetella, Legionella, Campylobacter, Francisella, Pasteurella, Yersinia, Bartonella, Bacteroides, Streptobacillus, Spirillum, Moraxella, and Shigella. In some embodiments gram-negative bacteria include Escherichia coli, Chlamydia trachomatis, Chlamydia caviae, Chlamydia pneumoniae, Chlamydia muridarum, Chlamydia psittaci, Chlamydia pecorum, Pseudomonas aeruginosa, Neisseria meningitides, Neisseria gonorrhoeae, Salmonella typhimurium, Salmonella entertidis, Klebsiella pneumoniae, Haemophilus influenzae, Haemophilus ducreyi, Proteus mirabilis, Vibro cholera, Helicobacter pylori, Brucella abortis, Brucella melitensis, Brucella suis, Bordetella pertussis, Bordetella parapertussis, Legionella pneumophila, Campylobacter fetus, Campylobacter jejuni, Francisella tularensis, Pasteurella multocida, Yersinia pestis, Bartonella bacilliformis, Bacteroides fragilis, Bartonella henselae, Streptobacillus moniliformis, Spirillum minus, Moraxella catarrhalis (Branhamella catarrhalis), and Shigella dysenteriae. In some embodiments, gram-negative bacteria include spirochetes including those belonging to the genera Treponema, Leptospira, and Borrelia. Particular spirochetes include, but are not limited to, Treponema palladium, Treponema pertenue, Treponema carateum, Leptospira interrogans, Borrelia burgdorferi, and Borrelia recurrentis. In some embodiments, gram-negative bacteria include those of the order Rickettsiales including those belonging to the genera Rickettsia, Ehrlichia, Orienta, Bartonella and Coxiella. In some embodiments, gram-negative bacteria include Rickettsia rickettsii, Rickettsia akari, Rickettsia prowazekii, Rickettsia typhi, Rickettsia conorii, Rickettsia sibirica, Rickettsia australis, Rickettsia japonica, Ehrlichia chaffeensis, Orienta tsutsugamushi, Bartonella quintana, and Coxiella burni.

In some embodiments, gram-positive bacteria include those of the genera Listeria, Staphylococcus, Streptococcus, Bacillus, Corynebacterium, Peptostreptococcus, Actinomyces, Propionibacterium, Clostridium, Nocardia, and Streptomyces. In some embodiments, gram-positive bacteria include Listeria monocytogenes, Staphylococcus aureus, Streptococcus pyogenes, Streptococcus pneumoniae, Bacillus cereus, Bacillus anthraci s, Clostridium botulinum, Clostridium perfringens, Clostridium difficile, Clostridium tetani, Corynebacterium diphtheriae, Corynebacterium ulcerans, Peptostreptococcus anaerobius, Actinomyces israeli, Actinomyces gerencseriae, Actinomyces viscosus, Actinomyces naeslundii, Propionibacterium propionicus, Nocardia asteroides, Nocardia brasiliensis, Nocardia otitidiscaviarum, and Streptomyces somaliensis.

In some embodiments, non-host nucleic acids are indicative of Mycobacteria such as, Mycobacterium tuberculosis, Mycobacterium leprae, Mycobacterium avium intracellulare, Mycobacterium kansasii, and Mycobacterium ulcerans. In some embodiments, non-host nucleic acids are indicative of Mycoplasma including, those of the genera Mycoplasma and Ureaplasma, such as Mycoplasma pneumoniae, Mycoplasma hominis, Mycoplasma genitalium, and Ureaplasma urealyticum.

In some embodiments, non-host nucleic acids are indicative of a fungus including those belonging to the genera Aspergillus, Candida, Cryptococcus, Coccidioides, Sporothrix, Blastomyces, Histoplasma, Pneumocystis, and Saccharomyces. In some embodiments, non-host nucleic acids are indicative of a fungus including Aspergillus fumigatus, Aspergillus flavus, Aspergillus niger, Aspergillus terreus, Aspergillus nidulans, Candida albicans, Coccidioides immitis, Cryptococcus neoformans, Sporothrix schenckii, Blastomyces dermatitidis, Histoplasma capsulatum, Histoplasma duboisii, and Saccharomyces cerevisiae.

In some embodiments, non-host nucleic acids are indicative of a parasitic cell including those belonging to the genera Entamoeba, Dientamoeba, Giardia, Balantidium, Trichomonas, Cryptosporidium, Isospora, Plasmodium, Leishmania, Trypanosoma, Babesia, Naegleria, Acanthamoeba, Balamuthia, Enterobius, Strongyloides, Ascaradia, Trichuris, Necator, Ancylostoma, Uncinaria, Onchocerca, Mesocestoides, Echinococcus, Taenia, Diphylobothrium, Hymenolepsis, Moniezia, Dicytocaulus, Dirofilaria, Wuchereria, Brugia, Toxocara, Rhabditida, Spirurida, Dicrocoelium, Clonorchis, Echinostoma, Fasciola, Fascioloides, Opisthorchis, Paragonimus, and Schistosoma. In some embodiments, non-host nucleic acids are indicative of a parasitic cell including Entamoeba histolytica, Dientamoeba fragilis, Giardia lamblia, Balantidium coli, Trichomonas vaginalis, Cryptosporidium parvum, Isospora belli, Plasmodium malariae, Plasmodium ovale, Plasmodium falciparum, Plasmodium vivax, Leishmania braziliensis, Leishmania donovani, Leishmania tropica, Trypanosoma cruzi, Trypanosoma brucei, Babesia divergens, Babesia microti, Naegleria fowleri, Acanthamoeba culbertsoni, Acanthamoeba polyphaga, Acanthamoeba castellanii, Acanthamoeba astronyxis Acanthamoeba hatchetti, Acanthamoeba rhysodes, Balamuthia mandrillaris, Enterobius vermicularis, Strongyloides stercoralis, Strongyloides fulleborni, Ascaris lumbricoides, Trichuris trichiura, Necator americanus, Ancylostoma duodenale, Ancylostoma ceylanicum, Ancylostoma braziliense, Ancylostoma caninum, Uncinaria stenocephala, Onchocerca volvulus, Mesocestoides variabilis, Echinococcus granulosus, Taenia solium, Diphylobothrium latum, Hymenolepis nana, Hymenolepis diminuta, Moniezia expansa, Moniezia benedeni, Dicytocaulus viviparous, Dicytocaulus filarial, Dicytocaulus arnfieldi, Dirofilaria repens, Dirofilaria immitis, Wuchereria bancrofti, Brugia malayi, Toxocara canis, Toxocara cati, Dicrocoelium dendriticum, Clonorchis sinensis, Echinostoma, Echinostoma ilocanum, Echinostoma j assyenese, Echinostoma malayanum, Echinostoma caproni, Fasciola hepatica, Fasciola gigantica, Fascioloides magna, Opisthorchis viverrini, Opisthorchis felineus, Opisthorchis sinensis, Paragonimus westermani, Schistosoma japonicum, Schistosoma mansoni, Schistosoma haematobium, and Schistosoma haematobium.

In some embodiments, non-host nucleic acids are indicative of a virus including those of the families Flaviviridae, Arenaviradae, Bunyaviridae, Filoviridae, Poxyiridae, Togaviridae, Paramyxoviridae, Herpesviridae, Picornaviridae, Caliciviridae, Reoviridae, Rhabdoviridae, Papovaviridae, Parvoviridae, Adenoviridae, Hepadnaviridae, Coronaviridae, Retroviridae, and Orthomyxoviridae. In some embodiments, non-host nucleic acids are indicative of a virus including Yellow fever virus, St. Louis encephalitis virus, Dengue virus, Hepatitis G virus, Hepatitis C virus, Bovine diarrhea virus, West Nile virus, Japanese B encephalitis virus, Murray Valley encephalitis virus, Central European tick-borne encephalitis virus, Far eastern tick-born encephalitis virus, Kyasanur forest virus, Louping ill virus, Powassan virus, Omsk hemorrhagic fever virus, Kumilinge virus, Absetarov anzalova hypr virus, Ilheus virus, Rocio encephalitis virus, Langat virus, Lymphocytic choriomeningitis virus, Junin virus, Bolivian hemorrhagic fever virus, Lassa fever virus, California encephalitis virus, Hantaan virus, Nairobi sheep disease virus, Bunyamwera virus, Sandfly fever virus, Rift valley fever virus, Crimean-Congo hemorrhagic fever virus, Marburg virus, Ebola virus, Variola virus, Monkeypox virus, Vaccinia virus, Cowpox virus, Orf virus, Pseudocowpox virus, Molluscum contagiosum virus, Yaba monkey tumor virus, Tanapox virus, Raccoonpox virus, Camelpox virus, Mousepox virus, Tanterapox virus, Volepox virus, Buffalopox virus, Rabbitpox virus, Uasin gishu disease virus, Sealpox virus, Bovine papular stomatitis virus, Camel contagious eethyma virus, Chamios contagious eethyma virus, Red squirrel parapox virus, Juncopox virus, Pigeonpox virus, Psittacinepox virus, Quailpox virus, Sparrowpox virus, Starlingpox virus, Peacockpox virus, Penguinpox virus, Mynahpox virus, Sheeppox virus, Goatpox virus, Lumpy skin disease virus, Myxoma virus, Hare fibroma virus, Fibroma virus, Squirrel fibroma virus, Malignant rabbit fibroma virus, Swinepox virus, Yaba-like disease virus, Albatrosspox virus, Cotia virus, Embu virus, Marmosetpox virus, Marsupialpox virus, Mule deer poxvirus virus, Volepox virus, Skunkpox virus, Rubella virus, Eastern equine encephalitis virus, Western equine encephalitis virus, Venezuelan equine encephalitis virus, Sindbis virus, Semliki forest virus, Chikungunya virus, O′nyong-nyong virus, Ross river virus, Parainfluenza virus, Mumps virus, Measles virus (rubeola virus), Respiratory syncytial virus, Herpes simplex virus type 1, Herpes simplex virus type 2, Varicella-zoster virus, Epstein-Barr virus, Cytomegalovirus, Human b-lymphotrophic virus, Human herpesvirus 7, Human herpesvirus 8, Poliovirus, Coxsackie A virus, Coxsackie B virus, Rhinovirus, Hepatitis A virus, Mengovirus, ME virus, Encephalomyocarditis (EMC) virus, MM virus, Columbia SK virus, Norwalk agent, Hepatitis E virus, Colorado tick fever virus, Rotavirus, Vesicular stomatitis virus, Rabies virus, Papilloma virus, BK virus, JC virus, B19 virus, Adeno-associated virus, Adenovirus, serotypes 3, 7, 14, 21, Adenovirus, serotypes 11, 21, Adenovirus, Hepatitis B virus, Coronavirus, Human T-cell lymphotrophic virus, Human immunodeficiency virus, Human foamy virus, Influenza viruses, types A, B, C, and Thogotovirus.

### Preparing Host-Specific Nucleic Acid Probes

Some embodiments of the methods and compositions provided herein include preparing host-specific nucleic acid probes, such as capture probes. In some embodiments, host-specific nucleic acid probes include aRNAs. In some embodiments, host-specific nucleic acid probes, such as capture probes are prepared from a source selected from the group consisting of a cell, a cell-line, a tissue, and an organ. In some embodiments, a plurality of host-specific nucleic acid probes , such as capture probes comprise capture probes complementary to RNAs selected from the group consisting of messenger RNAs, ribosomal RNAs, mitochondrial RNAs, transfer RNAs, micro RNAs, small RNAs, and small inhibitory RNAs.

There are a variety of methods to prepare aRNAs from target RNAs. In some embodiments, aRNAs may be prepared by linking an RNA polymerase promoter to a nucleic acid that has the sequence of the target RNA. In some embodiments, aRNAs are prepared by Eberwine amplification which includes a linear amplification method for preparing aRNA from target RNAs. (Phillips J. and Eberwine J. H., Methods 10:283-8, which is incorporated herein by reference in its entirety). Briefly, target RNAs are reverse transcribed, and a poly(T) primer modified 5′ with a T7 RNA polymerase promoter sequence is linked to a strand of the cDNAs. In some embodiments, the primer is a random hexamer modified 5′ with a T7 RNA polymerase promoter sequence. In some embodiments, the primer is a semi-random hexamer containing at least 1 ambiguous base that is modified 5′ with a T7 RNA polymerase promoter sequence. In some embodiments, the primer is a random nanomer modified 5′ with a T7 RNA polymerase promoter sequence. In some embodiments, the primer is a semi-random nanomer containing at least 1 ambiguous base that is modified 5′ with a T7 RNA polymerase promoter sequence. In one preferred embodiment, a combination of (i) a poly(T) primer modified 5′ with a T7 RNA polymerase promoter sequence and (ii) a semi-random nanomer containing at least 1 ambiguous base that is modified 5′ with a T7 RNA polymerase promoter sequence is used in the linear amplification method, leading to desired amplification of both polyadenylated and nonpolyadenylated RNAs. The cDNAs transcribed therefore contain the T7 promoter sequence. Following subsequent processing for second-strand cDNA synthesis, T7 RNA polymerase is used for amplification, which results in amplification of antisense RNA. More methods for generating aRNAs are also disclosed in Bak M. et al. (Anal Biochem 2006 Nov 1;358(1):111-9. Epub 2006 Sep 7) which is incorporated herein by reference in its entirety. More methods for generating aRNAs are described in U.S. Pat. Nos. 6,132,997, 5,545,522, 5,716,785, and 5,891,636, all of which are incorporated herein by reference.

In some embodiments, aRNAs prepared by any of the methods here described are fragmented to improve hybridization to target nucleic acids. In some embodiments, fragmentation is achieved by heating. In one preferred embodiment, RNA is fragmented by incubation at 95° C. for 80 minutes. It will be apparent to those skilled in the art that fragmentation may vary depending on the size or quality of the RNA source for aRNA preparation and the composition of the buffer used for fragmentation.

In some embodiments, aRNAs may be prepared by tailing and amplifying target RNAs. Some embodiments of such methods are provided in U.S. Pat. No. 7,361,465, which is incorporated herein in its entirety. As used herein, “tailing” or “tagging” a targeted RNA molecule with a nucleic acid tail means covalently binding a nucleic acid sequence to the targeted RNA molecule. In preferred embodiments, the nucleic acid sequence is covalently bound to the targeted RNA molecule enzymatically. The nucleic acid sequence tail may be added to an end of the targeted RNA molecule. In a specific embodiment, the nucleic acid tail is added to the 3′ end of the targeted RNA molecule. In some embodiments, the targeted RNA being amplified is poly(A)-tailed. In certain aspects, amplifying the poly(A)-tailed RNA comprises: hybridizing the poly(A)-tailed RNA with a promoter-oligo-dT primer; extending the promoter-oligo-dT primer using a reverse transcriptase to form a first strand DNA complementary to the poly(A)-tailed RNA; synthesizing a second strand DNA complementary to the first strand; and transcribing copies of RNA initiated from the promoter-oligo-dT primer using an RNA polymerase, wherein the RNA is complementary to the second strand DNA. The transcribed RNA represents the anti-sense RNA strand. In some embodiments, different RNA polymerase transcription start sites are appended on opposite ends to enable amplification of sense or antisense RNA as desired.

In some embodiments, the RNA polymerase may be, for example, a T bacteriophage RNA polymerase or an SP6 RNA polymerase. In some embodiments, the T bacteriophage RNA polymerase is T7 RNA polymerase or T3 RNA polymerase. In some embodiments, the reverse transcriptase is Moloney murine leukemia virus (MMLV) reverse transcriptase or avian myeloblastosis virus (AMV) reverse transcriptase. The reverse transcriptase may be a mutant reverse transcriptase, as long as the mutants retain cDNA synthesizing activity. Examples of reverse transcriptase mutants include those with reduced or no RnaseH activity (e.g., Superscript™ II, Superscript™ III, and ThermoScript™ (Invitrogen)) and those with enhanced activity at higher temperatures (Superscript™ III and ThermoScript™ (Invitrogen)). In some embodiments, higher temperatures during transcription are used to denature RNA secondary structure to permit longer transcripts. In one preferred embodiment the reverse transcriptase is Arrayscript™ (Ambion), which is a mutant MMLV with reduced RnaseH activity.

In some embodiments, the aRNAs are labeled. A number of different labels may be used in the present invention such as fluorophores, chromophores, radiophores, enzymatic tags, antibodies, chemiluminescence, and electroluminescence. Examples of fluorophores include, but are not limited to the following: Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy 3.5, Cy5, Cy5.5, Cy7, 6-FAM, Fluoroscein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, lissamine, phycoerythrin, FluorX, and Texas Red.

In some embodiments, affinity tags or affinity labels are linked and/or incorporated into aRNAs. Examples of affinity labels include an antibody, an antibody fragment, a receptor protein, a hormone, biotin, DNP, or any polypeptide/protein molecule that binds to an affinity label.

In some embodiments, preparing aRNAs include a random-primed reverse transcriptase-in vitro transcription (RT-IVT) method of linearly amplifying RNA. Examples of such methods are described in U.S. Pat. No. 7,229,765 which is incorporated herein by reference in its entirety. In some embodiments, target RNA is converted to double-stranded cDNA using two random primers, one of which comprises a RNA polymerase promoter sequence (“promoter-primer”), to yield a double-stranded cDNA that comprises a RNA polymerase promoter that is recognized by a RNA polymerase In some embodiments, the primer for first-strand cDNA synthesis is a promoter-primer and the primer for second-strand cDNA synthesis is not a promoter-primer. The double-stranded cDNA is then transcribed into RNA by the RNA polymerase, optimally in the presence of a reverse transcriptase that is rendered incapable of RNA-dependent DNA polymerase activity during this transcription step.

In some embodiments, a plurality of capture probes comprise capture probes prepared by a method that includes (i) obtaining single-stranded target nucleic acids; (ii) obtaining double-stranded target nucleic acids from the single-stranded target nucleic acids, wherein the double-stranded target nucleic acids comprise an RNA polymerase promoter; and (iii) contacting the double-stranded nucleic acids with an RNA polymerase to obtain RNAs complementary to the single-stranded target nucleic acids.

In some embodiments, the double-stranded target nucleic acids comprise cDNA. In some embodiments, obtaining double-stranded target nucleic acids from the single-stranded target nucleic acids comprises linking the double-stranded target nucleic acids with a primer comprising the RNA polymerase promoter or complement thereof. Embodiments of such methods are disclosed in U.S. Pat. No. 7,229,765, which in incorporated herein by reference in its entirety. In some embodiments, the double-stranded target nucleic acids comprise RNA. In some embodiments, obtaining double-stranded target nucleic acids from the single-stranded target nucleic acids comprises linking the single-stranded target nucleic acids with a primer comprising the RNA polymerase promoter or a complement thereof. Embodiments of such methods are disclosed in U.S. Pat. No. 7,361,465, which in incorporated herein by reference in its entirety. In some embodiments, obtaining double-stranded target nucleic acids from the single-stranded target nucleic acids comprises linking the single-stranded nucleic acids with an adapter primer, and hybridizing a primer comprising the RNA polymerase promoter or a complement thereof to the adapter primer. In some embodiments, obtaining double-stranded target nucleic acids from the single-stranded target nucleic acids comprises contacting the single-stranded target nucleic acids with a reverse transcriptase. In some embodiments, the reverse transcriptase is selected from the group consisting of Moloney murine leukemia virus (MMLV) reverse transcriptase, and avian myeloblastosis virus (AMV) reverse transcriptase.

In some embodiments, a plurality of capture probes comprises capture probes prepared by a method comprising: (i) fragmenting double-stranded nucleic acids; (ii) linking the double-stranded nucleic acids fragments with a primer comprising an RNA polymerase promoter; and (iii) amplifying the double-stranded nucleic acids fragments with an RNA polymerase. Embodiments of such methods are disclosed in U.S. 2013/0230857, which in incorporated herein by reference in its entirety. In some embodiments, the double-stranded nucleic acids comprise genomic DNA.

In some embodiments, a plurality of capture probes comprises capture probes prepared by a method comprising: (i) inserting a plurality of transposons into target nucleic acids, wherein insertion of transposons by the transposome complex simultaneously fragments the DNA and inserts an RNA polymerase promoter into the target nucleic acids; and (ii) amplifying the double-stranded nucleic acids fragments with an RNA polymerase. In some embodiments, the transposon is selected from the group consisting of Mu, Mu E392Q, Tn5, RAG, and Tn552.

In some embodiments, the RNA polymerase is selected from the group consisting of T7 RNA polymerase, T3 RNA polymerase, and SP6 RNA polymerase.

In some embodiments, the plurality of capture probes comprises capture probes prepared by amplification of RNA to obtain the capture probes comprising affinity tags. In some embodiments, the affinity tag is selected from the group consisting of an antibody, an antibody fragment, a receptor protein, a hormone, biotin, streptavidin, a His tag, and digoxin.

### Compositions, Kits, and Systems

Some embodiments of the methods and compositions provided herein include compositions, kits, and systems for enrichment of non-host nucleic acids, such as non-host RNAs in a nucleic acid sample. In some embodiments, kits can include reagents for preparing capture probes, such as aRNA; reagents for preparing capture probes comprising affinity tags; and/or reagents for depleting host nucleic acids, such as host DNA, such as host RNAs, such as host polyadenylated RNAs, ribosomal RNAs, tRNAs, mitchondial RNAs, and other host non-polyadenylated RNAs such as various other noncoding or coding RNAs that lack a polyA tail. In some embodiments, systems can include the preparation of capture probes, such as aRNAs; the acquirement of sequencing information from a host-depleted nucleic acids sample; and/or the determination of the presence or absence of certain non-host nucleic acids in the depleted nucleic acid sample.

### EXAMPLES

### Example 1

### Enrichment of Non-Host RNAs

This example illustrates an embodiment for the preparation of antisense RNA (aRNA) capture probes. Biotinylated probes complementary to human RNA including ribosomal, mitochondrial, messenger, and non-coding RNAs were prepared and hybridized to samples comprising human RNAs and non-human RNAs. The probes were removed to provide an enriched sample of non-human RNAs. In addition, the method also included the removal of other human RNAs such as ribosomal and mitochondrial RNAs.

Generation of biotinylated capture probes. Capture probes were prepared from human RNA by Eberwine amplification using a TargetAmp™ kit (Epicentre Technologies Corp., Madison, Wis.). The TargetAmp™ kit produces aRNA from cellular RNA. The aRNA was labeled by incorporation of biotin-conjugated UTP using a TargetAmp™ kit nano labeling kit (Epicentre Technologies Corp., Madison, Wis.) with the addition of a semi-random hexamer modified 5′ with a T7 RNA polymerase promoter. From 200 ng input RNA, approximately 50-75 μg labeled aRNA probes were generated. Probes were fragmented by incubating for 80 minutes at 95° C.

Hybridization of biotinylated probes to sample RNA. The following components were mixed in a total volume of 20 μl: 50 ng RNA sample containing host and non-host RNAs; 6 μl rRNA removal 10× reaction buffer (Ribo-Zero™ rRNA removal kit; Epicentre, Madison Wis.); 1 μl Ribo-Zero rRNA removal solution which includes biotinylated probes targeting mitochondrial RNA and rRNA from human and microbes; 2 μg fragmented biotinylated capture probes; 1 μl biotin-conjugated oligo-dT (Promega catalog #Z5261); and water. The foregoing was incubated for 10 minutes at 68° C. followed by 5 minutes at room temperature.

Removal of hybridized RNA and excess probe. A Ribo-Zero™ rRNA removal kit (Epicentre, Madison, Wis.) was used to remove additional host RNAs. This step followed the Ribo-Zero™ protocol, except after washing magnetic beads; the product was resuspended in 35 μl resuspension buffer. Briefly, for each sample, 225 μl magnetic streptavidin-coated beads were aliquoted in a 1.7 ml microfuge tube (all materials from Ribo-Zero™ magnetic gold epidemiology kit (Epicentre, Madison Wis.). The beads are washed twice with water, resuspended in 35 μl resuspension buffer to which 0.5 μl RiboGuard RNase inhibitor (Epicentre, Madison Wis.) was added. Beads were removed by transfer to a magnetic stand for 1 minute, and the supernatant was reserved. The RNA was further processed with an AMPure, Purification system (Beckman Coulter). The RNA was eluted in 12 μl water and used for RNAseq library preparation.

### Example 2

### Enrichment of Non-Host RNAs

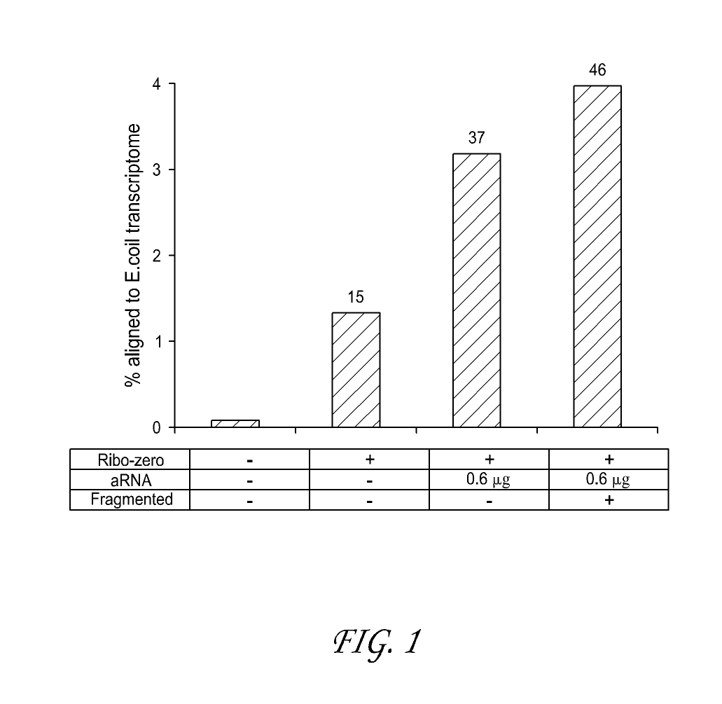

RNA samples were obtained that included host RNAs with non-host E. coli RNAs. RNAs were prepared using (1) Ribo-Zero™ kit (Epicentre, Madison, Wis.) only which removes mitochondrial RNA and rRNA from human and rRNA from bacteria; (2) Ribo-Zero™ kit with the protocol of Example 1 which includes the use of prepared biotinylated capture probes (aRNA) prepared by the method of Example 1; (3) Ribo-Zero™ kit with the protocol of Example 1 which includes the use of prepared biotinylated capture probes (aRNA), that were heat fragmented to improve hybridization; and (4) control with no preparation of RNA sample. The products were sequenced and obtained E. coli sequences aligned to the E. coli transcriptome. The fold enrichment of the non-host E. coli RNAs were measured compared to the control. The results are shown in FIG. 1. A 46-fold enrichment was observed in sample (3) in which the sample had been treated with the Ribo-Zero™ kit which targeted mitochondrial RNA and rRNA, and with the aRNA, and fragmentation.

### Example 3

### Sequencing Enriched RNA Samples

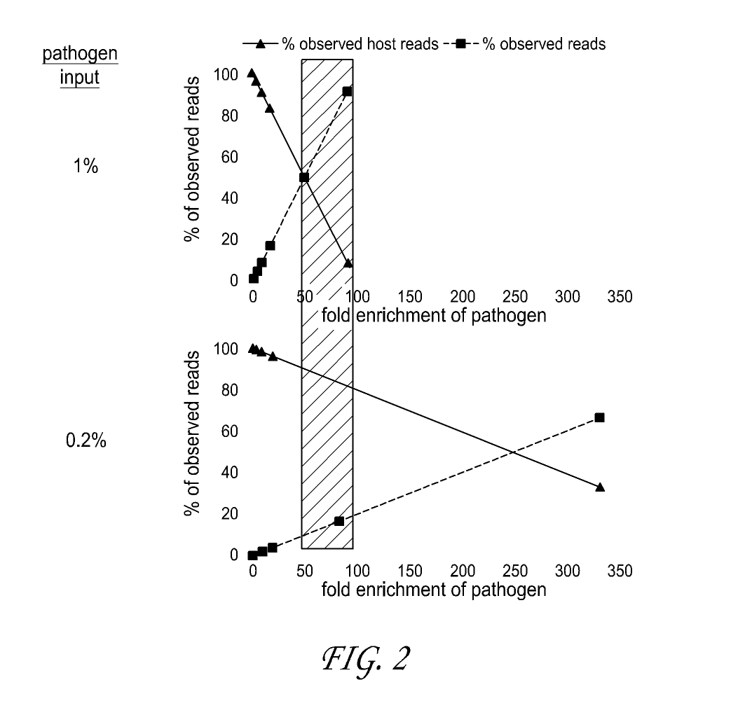

RNA samples containing 0.2% non-host RNAs or 1% non-host RNAs were prepared and non-host RNAs were enriched by a method similar to that described in Example 1. Enriched samples were sequenced. FIG.2 depicts a calculation of the level of enrichment needed to give the indicated percent of observed pathogen reads for samples starting with 0.2% or 1.0% non-host RNA, respectively

### Example 4

### Enrichment of Non-Host RNAs

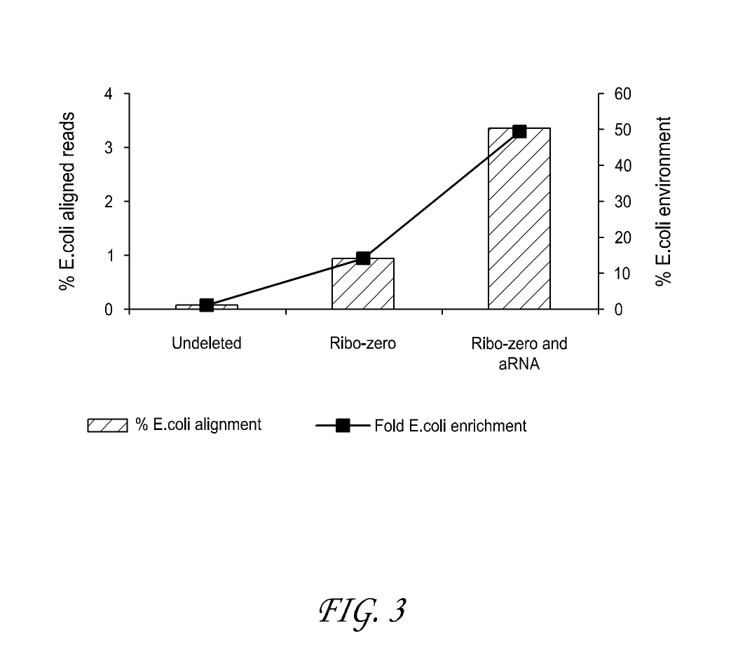

RNA samples comprising Universal Human Reference RNA (host) (human RNA from 10 different cancer cell lines, Agilent) and E. coli RNA (non-host) were prepared. Host RNAs were depleted from the samples using either Ribo-Zero alone, or Ribo-Zero in combination with aRNA probes prepared by the method of Example 1. RNA-seq libraries were prepared by ScriptSeq (Epicentre, Madison, Wis.). Percentage alignment and fold enrichment of non-host RNAs were determined and are shown in FIG. 3.

With regards to sequencing information, it was found that there was an unexpected enrichment of non-coding RNAs after depletion with Ribo-Zero probes in combination with aRNA probes. With Ribo-Zero depletion alone, 7 out of the top 50 highest expressed transcripts were noncoding RNA, the remainder were for coding transcripts. With the combination probe depletion, 48 out of the top 50 highest expressed transcripts were noncoding RNA, the remainder were for coding transcripts.

### Example 5

### Enrichment of Non-Host RNAs

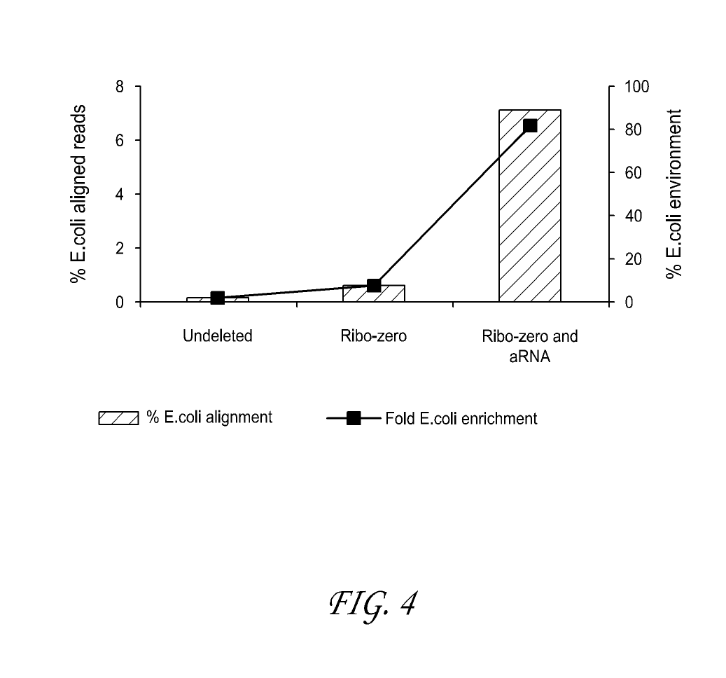

Samples comprising Human RNA from CD4-positive helper T cells (Miltenyi Biotec) and E. coli RNA (Ambion) were prepared. Human RNA was depleted from the samples using either Ribo-Zero alone, or Ribo-Zero in combination with aRNA probes prepared by the method of Example 1. RNA-seq libraries were prepared by ScriptSeq (Epicentre, Madison, Wis.). Percentage alignment and fold enrichment of non-host RNAs were determined and are shown in FIG. 4. Table 1 provides the results of Examples 4 and 5.

TABLE 1

| | | Aligned coding | | |
|---|---|---|---|---|
| | | reads (%) | | Fold E. coli |
| Sample | Treatment | Human | E. coli | enrichment |
| Universal human | undepleted | 18.7 | 0.06 | 1.0 |
| reference RNA | Ribo-zero | 86.4 | 0.9 | 14.7 |
| | Ribo zero with | 81.3 | 3.4 | 53.1 |
| | aRNA | | | |
| CD4+ T cell | undepleted | 20.1 | 0.08 | 1.0 |
| RNA | Ribo-zero | 89.8 | 0.6 | 7.4 |
| | Ribo zero with | 75.8 | 7.1 | 84.2 |
| | aRNA | | | |

### Example 6

### Enrichment of Non-Host RNAs

RNA samples comprising human RNAs from blood (host) and plasmodium RNAs (non-host) were prepared. Host RNAs were depleted from the samples using either Ribo-Zero alone, or Ribo-Zero in combination with aRNA probes prepared by the method of Example 1. RNA-seq libraries were prepared by ScriptSeq (Epicentre, Madison, Wis.). Percentage alignment and fold enrichment of non-host RNAs were determined are shown in Table 2. Aligned reads for both human and plasmodium RNAs increase with Ribo-Zero treatment alone, as rRNA is removed, while only plasmodium reads are further enriched by the combination of Ribo-Zero and aRNA probes.

TABLE 2

Aligned reads (%)

| Sample | Treatment | Human | Plasmodium |
|---|---|---|---|
| 346P09 | undepleted | 13.4 | 9.2 |
| | Ribo-zero | 60.5 | 24.1 |
| | Ribo zero with | 53.5 | 30.7 |
| | aRNA | | |
| 100VI | undepleted | 25.7 | 8.2 |
| | Ribo-zero | 79.5 | 8.9 |
| | Ribo zero with | 77.8 | 12.6 |
| | aRNA | | |

### Example 7

### Enrichment of Non-Host RNAs

Various RNA samples comprising human RNAs from blood (host) (human RNA from 10 different cancer cell lines, Agilent) and various amounts of E. coli RNAs (non-host) were prepared. From 100 ng of total human/E. coli mixed RNA, host RNAs were depleted from the samples using Ribo-Zero in combination with aRNA probes prepared by the method of Example 1. RNA-seq libraries of the dehosted samples were prepared by ScriptSeq (Epicentre, Madison, Wis.). The number of E-coli gene sequences for each sample was determined and is shown in Table 3 and Table 4.

TABLE 3

| | E. coli RNA spiked in | | | | |
|---|---|---|---|---|---|
| Treatment | None | 0.01% | 0.30% | 1% | 3% |
| No depletion | 265 | 301 | 534 | 550 | 648 |
| Ribo zero | | 726 | 845 | 860 | 891 |
| with aRNA | | | | | |

# gene isoforms detected at >100 counts per 60 million reads

TABLE 4

| | E. coli RNA spiked in | | | | |
|---|---|---|---|---|---|
| Treatment | None | 0.01% | 0.30% | 1% | 3% |
| No depletion | 0 | 36 | 269 | 285 | 383 |
| Ribo zero | | 461 | 580 | 595 | 626 |
| with aRNA | | | | | |

# gene isoforms detected at >100 counts per 60 million reads (negative control subtracted)

Depletion by the combination probe methodology described herein increased the number of E. coli genes detected (100 counts per 60 million reads was arbitrarily chosen as cutoff). Even in the absence of spiked-in E. coli RNA, a subset of reads aligned to E. coli (Table 3, “none”). Preliminary analysis indicated that some of these sequences include many bases called as “N”. Table 4 displays the same data, subtracting these putative ‘junk’ sequences. Notably, as Table 4 illustrates, more non-host sequences were detected in the 0.01% initial E. coli RNA amount (461 sequences) after enrichment, compared to the 3% initial E. coli RNA amount (383 sequences) before enrichment, demonstrating greater than 300 fold increase in sensitivity for non-host gene detection.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A method for the enrichment of non-host RNAs in a nucleic acid sample comprising host RNAs and non-host RNAs, comprising:
(a) obtaining a plurality of capture probes, wherein each capture probe comprises an affinity tag and a nucleic acid complementary to a host RNA;
(b) contacting the nucleic acid sample with the plurality of capture probes; and
(c) removing capture probes hybridized to the host RNAs, thereby obtaining a population of nucleic acids enriched for non-host RNAs.

2. The method of claim 1, wherein the plurality of capture probes comprises capture probes prepared by a method comprising:
(i) obtaining single-stranded target nucleic acids;
(ii) obtaining double-stranded target nucleic acids from the single-stranded target nucleic acids, wherein the double-stranded target nucleic acids comprise an RNA polymerase promoter; and
(iii) contacting the double-stranded nucleic acids with an RNA polymerase to obtain RNAs complementary to the single-stranded target nucleic acids.

3. (canceled)

4. The method of claim 2, wherein step (ii) comprises linking the double-stranded target nucleic acids with a primer comprising the RNA polymerase promoter or complement thereof.

5. (canceled)

6. The method of claim 2, wherein step (ii) comprises linking the single-stranded target nucleic acids with a primer comprising the RNA polymerase promoter or a complement thereof.

7. The method of claim 2, wherein step (ii) comprises linking the single-stranded nucleic acids with an adapter primer, and hybridizing a primer comprising the RNA polymerase promoter or a complement thereof to the adapter primer.

8. The method of claim 2, wherein step (ii) comprises contacting the single-stranded target nucleic acids with a reverse transcriptase.

9. (canceled)

10. The method of claim 1, wherein the plurality of capture probes comprises capture probes prepared by a method comprising:
(i) linking the double-stranded nucleic acids fragments with a primer comprising an RNA polymerase promoter;
(ii) amplifying the double-stranded nucleic acids fragments with an RNA polymerase; and
(iii) fragmenting the RNA probes.

11. (canceled)

12. The method of claim 1, wherein the plurality of capture probes comprises capture probes prepared by a method comprising:
(i) inserting a plurality of transposons into target nucleic acids, wherein insertion of the transposon into target nucleic acid by the transposome complex fragments the target nucleic acid and simultaneously inserts an RNA polymerase promoter; and
(iii) amplifying the double-stranded nucleic acids fragments with an RNA polymerase.

13. The method of claim 12, wherein the transposon is selected from the group consisting of Mu, Mu E392Q, Tn5, RAG, and Tn552.

14. The method of claim 12, wherein the transposons comprise a fragmentation site, said method further comprising fragmenting the target nucleic acid at the fragmentation sites.

15. (canceled)

16. The method of claim 1, wherein the plurality of capture probes comprises capture probes prepared by amplification to obtain the capture probes comprising affinity tags, wherein the affinity tag is optionally selected from the group consisting of an antibody, an antibody fragment, a receptor protein, a hormone, biotin, streptavidin, a His tag, and digoxin.

17. (canceled)

18. The method of claim 1, wherein the nucleic acid sample further comprises DNA.

19. The method of claim 18, further comprising depleting DNA from the nucleic acid sample.

20. The method of claim 1, further comprising depleting polyadenylated RNAs from the nucleic acid sample.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. The method of claim 1, wherein the plurality of capture probes is linked to a substrate.

35. (canceled)

36. (canceled)

37. The method of claim 1, wherein the plurality of capture probes is in solution.

38. The method of claim 1, wherein the non-host RNAs are enriched in the population of nucleic acids enriched for non-host RNAs compared to the nucleic acid sample by at least 10-fold.

39. (canceled)

40. (canceled)

41. (canceled)

42. (canceled)

43. A nucleic acid sequencing library comprising nucleic acids obtained by the method of claim 1.

44. A method for detecting the presence of a pathogen in a sample comprising:
obtaining a nucleic acid sample comprising host RNAs and non-host RNAs from the sample, wherein the pathogen comprises the non-host RNAs;
enriching the nucleic acid sample for the non-host RNAs according to the method of claim 1; and
detecting the presence of the non-host RNAs in the enriched nucleic acid sample.

45. (canceled)

46. The method of claim 1, wherein host RNA depletion preferentially removes highly expressed host RNAs, therefore remaining host RNA is normalized in which non-coding RNAs and low expressors are preferentially enriched.
